(19) United States
(12) Patent Application Publication
(10) Pub. No.: US 2003/0049613 A1
(43) Pub. Date: Mar. 13, 2003

(54) METHODS FOR IDENTIFYING SOMATIC CHANGES IN GENOMIC SEQUENCES USEFUL FOR CANCER DIAGNOSIS AND PROGNOSIS

(76) Inventors: MANUEL PERUCHO, SAN DIEGO, CA (US); SERGEI MALKHOSYAN, SAN DIEGO, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/375,246

(22) Filed: Aug. 16, 1999

Related U.S. Application Data
(60) Provisional application No. 60/096,828, filed on Aug. 17, 1998.

(51) Int. Cl. C12Q 1/68

(52) U.S. Cl. 435/6

(57) ABSTRACT

A method of determining the clinical outcome of a subject with a cancer using a Genomic Damage Fraction comprising, (a) determining the relative change in quantity of nucleic acids between cancerous cells and non-cancerous cells of said subject, (b) determining the Genomic Damage Fraction from the results of step (a), and (c) determining the prognosis of said subject according to said subject's GDF, where a GDF greater than a predetermined GDF is indicative of a first clinical outcome (e.g., a poor prognosis), and a GDF lesser than a predetermined GDF is indicative of a second clinical outcome (e.g., a good prognosis); and a method of identifying certain genomic sequences whose alterations during tumorigenesis of a subject with a cancer have prognostic value for determining the clinical outcome of said subject comprising, (a) determining the molecular profiles of genomic losses and gains (“amplotyping”) of tumors at different stages of progression from the same cancer patient, (b) identifying changes (losses and gains) specifically associated to the more advanced stages of tumor progression (e.g., metastatic stage), and (c) determining the prognosis of said subject according to said subject's status of these genomic sequences of step b, where a change (loss or gain) is indicative of a first clinical outcome (i.e., poor prognosis), and no change (i.e., no loss or gain) is indicative of a second clinical outcome (i.e., good prognosis).

This application claims the benefit of U.S. Provisional Application No. 60/096,828, filed Aug. 17, 1998, and is incorporated herein by reference.

This invention was made with government support under National Institutes of Health Grants CA38579 and CA63585.

### FIELD OF THE INVENTION

This invention relates generally to the fields of biochemistry, molecular genetics and medicine, and more specifically to methods for identifying genomic changes occurring in cancerous cells for the molecular diagnostics of cancer, and methods of determining the prognostic clinical outcome of subjects with cancer.

### BACKGROUND OF THE INVENTION

Genomic instability characterizes neoplastic transformation and generate tumor cell aneuploidy. Mutations activate positive regulators of cell growth or survival and inactivate factors with a negative role in these processes. Losses of heterozygosity (LOH) unmask recessive mutations in tumor suppressor genes. LOH can be detected by restriction fragment length polymorphisms (RFLP) of polymorphic minisatellite loci, and more recently by PCR amplification of highly informative microsatellite loci. The “allelotype” approach was critical for the identification and subsequent characterization of RB and p53 and the emergence of the tumor suppressor gene era.

But losses of genetic material provide only half of the picture of the distorted genome found in the majority of cancer cells. Chromosomal gains can be diagnostic indicators of the presence of dominant oncogenes such as c-K-ras. Gains of genetic material may also lead to overexpression of genes contributing to tumor progression in the absence of mutation. Thus, moderate gains (e.g., equivalent to trisomy/tetrasomy) of chromosomal fragments have been long known to be germane to neoplasia. The detection of such moderate chromosomal changes has been a challenge in cancer research, and perhaps as a consequence, “the pathogenetic significance of such abnormalities is totally unknown” as Mitelman et al., wrote in their recent review.

Recent progress in the molecular genetics of cancer has facilitated the detection of allelic abnormalities at the subchromosomal level. Representation differential analysis (RDA) is a powerful technique for the identification and isolation of sequences under and over-represented in tumor genomes. LOH analysis by RFLP or microallelotyping procedures can be used for the detection of tumor suppressor genes. However, these techniques cannot identify moderate gains of genetic material. Improvements to make PCR quantitative have been implemented for microallelotyping, but at the expense of losing simplicity. Comparative genomic hybridization (CGH) has allowed the assessment of numerical and structural chromosome aberrations. However, CGH requires special instrumentation and can only detect alterations of relatively large chromosomal regions.

DNA fingerprinting of polymorphic minisatellites has been used to study anonymous somatic mutations during tumorigenesis, either by one or two dimensional gel electrophoresis. However, these techniques utilize Southern blot hybridization of genomic DNA and the subsequent isolation and characterization of the altered sequences is difficult.

The Arbitrarily Primed Polymerase Chain Reaction or AP-PCR, as described in U.S. Pat. No. 5,487,985, is a PCR based DNA fingerprinting technique using single primers of arbitrarily chosen sequence and several initial cycles of low stringency. Primer annealing at multiple sites generates many PCR products that represent a DNA fingerprint specific for each primer-DNA template combination. Comparison of the AP-PCR fingerprints from matched tumor and normal tissues identifies somatic mutations. AP-PCR DNA fingerprinting was instrumental for the identification of the microsatellite mutator phenotype pathway for cancer. The detection of recurrent fingerprint band shifts revealed the tumor-specific accumulation of hundreds of thousands of somatic clonal mutations. This genome-wide instability in repetitive sequences underlies a mutator phenotype pathway for some sporadic and hereditary gastrointestinal cancers.

The quantitative nature of AP-PCR fingerprinting also allows the detection of allelic losses and gains in tumor cells by the reduction or increase in intensity of tumor fingerprint bands, respectively. The chromosomal origins of most fingerprint bands can be assigned simultaneously by AP-PCR of somatic monochromosome cell hybrid panels. These features offer an excellent opportunity to use AP-PCR DNA fingerprinting as an unbiased molecular karyotyping of tumors.

Evidence supporting the role in tumor development of moderate gains of genetic material is well established (Rabinowitz, Z. & Sachs, L., (1970) Nature (London) 225, 136-139; Spira, J., Wiener, F., Ohno, S. & Klein, G. (1979) Proc. Natl. Acad. Sci. USA 76, 6619-6621; and Klein, G. (1981) Nature (London) 294, 313-318). However, these moderate gains in cancer development have remained essentially unexplored. This is due in part to fashionable changes in the prevalent scientific archetypes: first, there was the excitement of the studies on mutant dominant oncogenes, and later on recessive tumor suppressor genes. These temporal fluctuations depends heavily on the technical advances preceding (Nakamura, Y., Leppert, M., O'Connell, P., Wolff, R., Holm, T., Culver, M., Martin, C., Fujimoto, E., Hoff, M., Kumlin, E., et al., (1987) Science 234, 1616-1622; and Wigler, M., Pellicer, a., Silverstein, S. & Axel, R. (1978) Cell 14, 725-731) the trend-setting studies (Vogelstein, B., Fearon, E. R., Kern, S. E., Hamilton, S. R., Preisinger, A. C., Nakamura, Y. & White, R. (1989) Science 244, 207-211; and Shih, C., Shilo, B. Z., Goldfarb, M. P., Dannenberg, A. & Weinberg, R. A. (1979) Proc. Natl. Acad. Sci. USA 76, 5714-5718). Although several approaches have been applied to study genetic changes in cancer cells, no simple techniques are available for the study of moderate gains of genetic material.

Despite the progress made in developing powerful analytical tools for examining the molecular genetics of cancer, such as, AP-PCR DNA fingerprinting, there remains an ongoing need to develop better methods for determining the prognostic clinical outcome of a subject with cancer, which would provide the health care provider with a much needed tool in prescribing the most advantageous course of treatment for such a subject with cancer.

The present invention satisfies this ongoing need and provides additional advantagous aspects as well. It has been surprisingly discovered, that applying AP-PCR DNA fingerprinting to study the prevalence of allelic losses and gains at different stages of colorectal tumor progression, a general method of determining the prognostic clinical outcome of a subject with cancer has been invented.

### SUMMARY OF THE INVENTION

A method of determining the clinical outcome of a subject with a cancer using a Genomic Damage Fraction comprising, (a) determining the relative change in quantity of nucleic acids between cancerous cells and non-cancerous cells of said subject, (b) determining the Genomic Damage Fraction from the results of step (a), and (c) determining the prognosis of said subject according to said subject's GDF, where a GDF greater than a predetermined GDF is indicative of a first clinical outcome (e.g., a poor prognosis), and a GDF lesser than a predetermined GDF is indicative of a second clinical outcome (e.g., a good prognosis); and a method of identifying certain genomic sequences whose alterations during tumorigenesis of a subject with a cancer have prognostic value for determining the clinical outcome of said subject comprising, (a) determining the molecular profiles of genomic losses and gains (“amplotyping”) of tumors at different stages of progression from the same cancer patient, (b) identifying changes (losses and gains) specifically associated to the more advanced stages of tumor progression (e.g., metastatic stage), and (c) determining the prognosis of said subject according to said subject's status of these genomic sequences of step b, where a change (loss or gain) is indicative of a first clinical outcome (i.e., poor prognosis), and no change (i.e., no loss or gain) is indicative of a second clinical outcome (i.e., good prognosis).

### BRIEF DESCRIPTION OF THE DRAWINGS

FIG. 1. AP-PCR DNA fingerprints of colorectal tumors. Autoradiogram of a denaturing polyacrylamide sequencing gel of the AP-PCR fingerprints generated by arbitrary primer MCG1 with 100 ng of genomic DNA isolated from normal and tumor tissues from colorectal cancer patients indicated at the top. The first, second and third fingerprint lanes of each case corresponds to normal tissue (N), primary tumor (P) and liver metastasis (M). N, P and M were available from patients # 1-12, and only N and M were available for patients # 13-15. Numbers at the left indicate the chromosomal origin of the bands named by letters at the right. Cases 1, 3, 6, 8, 10 and 12 were males. Band F (designated with an asterisk) was a composite of at least three sequences from chromosomes 2, 11, and 22. Some other bands were composite of sequences mainly derived from at least two chromosomes, such as bands C, L and N (Yasuda, J., Navarro, M., Malkhosyan, S., Velazquez, A., Seikya, T. & Perucho, M. (1996) Genomics 34, 1-7). In these cases, no estimation of intensity variations were attempted, except for band C (see Detailed Description). Some double bands represent the two strands of the same DNA molecule (such as bands B0, B2, G and J). Band S represents a length polymorphism that resolves the two alleles by their different size in heterozygous cases (cases 4, 5, 9, 10, 13 and 14). The approximate size of some of the cloned fingerprint bands are: D:800 bp; E:750 bp; F:710 bp; J:575; M:525 bp; and Q:405 bp.

FIG. 2. AP-PCR DNA fingerprints of tumor cell lines of characterized karyotypes. The cell lines and their gender are indicated at the top. Fingerprints were generated using 40 and 60 ng of DNA each cell line using primer MCG1. The band names are at right and the chromosomes of each band at left. Bands G and J, from chromosomes X and 13 are double bands because the two DNA strands are resolved in these denaturing gels. Band I (chromosome 7) is polymorphic in the human population, including length polymorphisms, and there are non-linear fluctuations in the intensity of the amplified PCR product due to sequence changes in the primer annealing regions. However, comparative analysis of tumor and normal tissues from the same individual is informative for gains or losses of these sequences, since the problem of inter-individual variation is eliminated. These cell lines were chosen because of their (pseudo)diploid nature (all but HT29). SW48 exhibits trisomy of 7, there is only one N13 in HuTu80 cells and LS174-T is monosomic for X (American Tissue Cell Type Collection).

FIG. 3. Microallelotyping of colorectal tumors. Microsatellite repeats D13S160 and D13S221 (from chromosome 13q) were amplified by PCR from some of the same genomic DNAs which were used in the experiments of FIG. 1 (cases indicated at the top). The radioactive PCR products were analyzed in denaturing sequencing gels.

FIG. 4. Chromosome 13 regions of gains and losses in metastatic colon cancer.

The graph depicts chromosome 13 with the position of the Rb locus and the three amplotype bands from the MCG1 primer (A₀, E and J) determined by PCR of radiation hybrid panels (Navarro et al, in preparation), and the dinucleotide repeats analyzed with their approximate localization. Triple bars summarize at right our analysis by amplotyping (left bars), microalleotyping (middle bars) and the combined analysis (right bars), for the metastatic tumors shown at the top. The summary of our studies, including other tumors not shown, is represented with a single bar under “Common regions” in the center of the figure. At the left of the figure is the summary of chromosomal changes observed by CGH (Ried, T., Knutzen, R., Steinbeck, R., Blegen, H., Schrock, E., Heselmeyer, K., du Manoir, S. & Auer, G., (1996) Genes Chromosomes Cancer 15, 234-245), where the thick lines represent chromosomal gains.

FIG. 5. Molecular karyotype (amplotype) of metastatic colon cancer.

Each bar represents the percent (among the 25 analyzed cancer cases) of loss (lower panel) or gain (upper panel) of a chromosomal region detected as a change of the intensity of a corresponding AP-PCR band. For instance, the three bands from chromosome 8 (bands D and O of MCG1 primer and band K from BLUE primer) are represented by three bars on chromosome 8. The data is derived from complete analysis of the fingerprints obtained with two primers, MCG1 and BLUE. A partial analysis of two bands from primer F generated information on the imbalance status of chromosomes 17 and 18.

FIG. 6. Comparative amplotypes of primary and metastatic colon cancer.

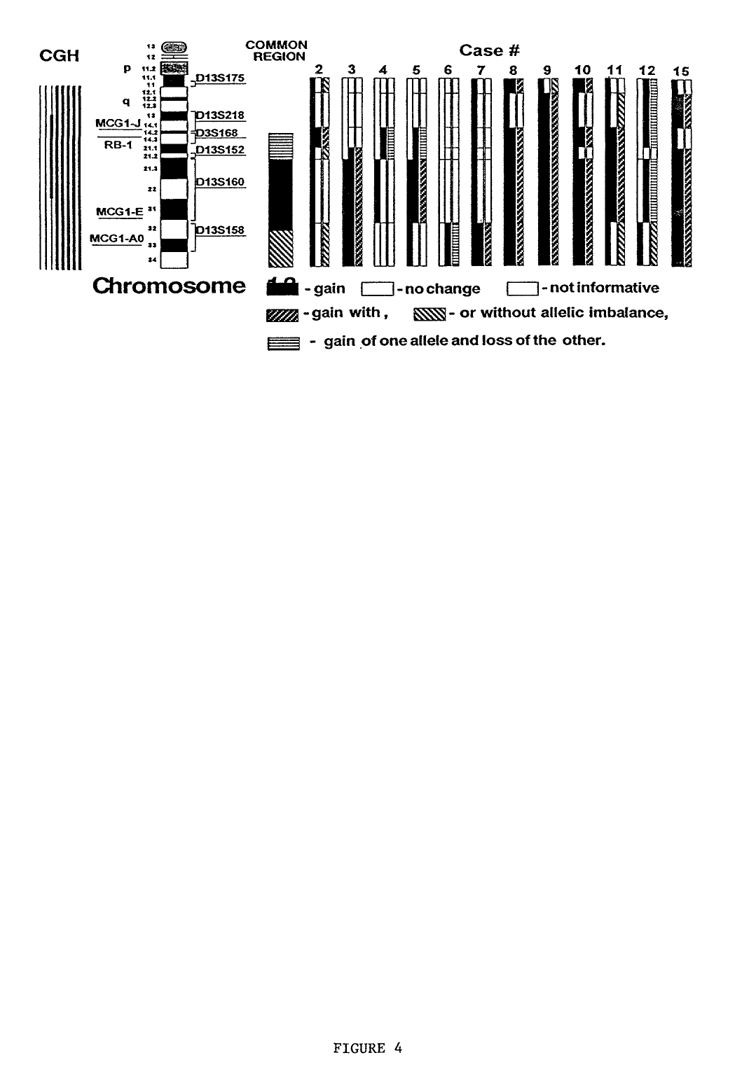

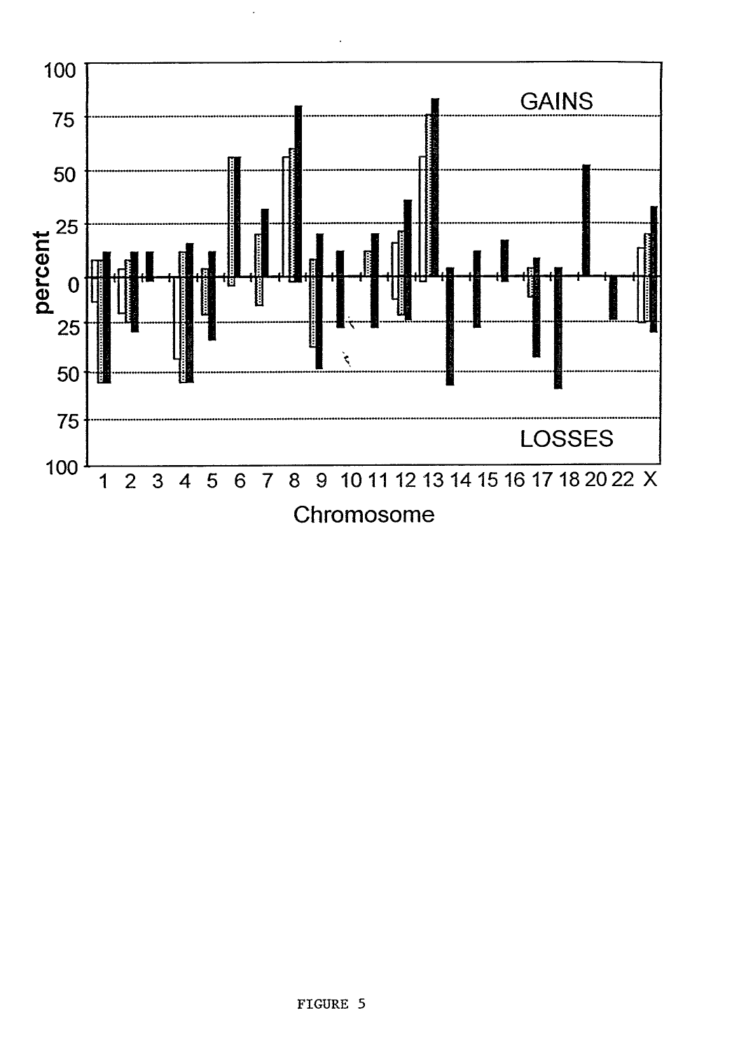

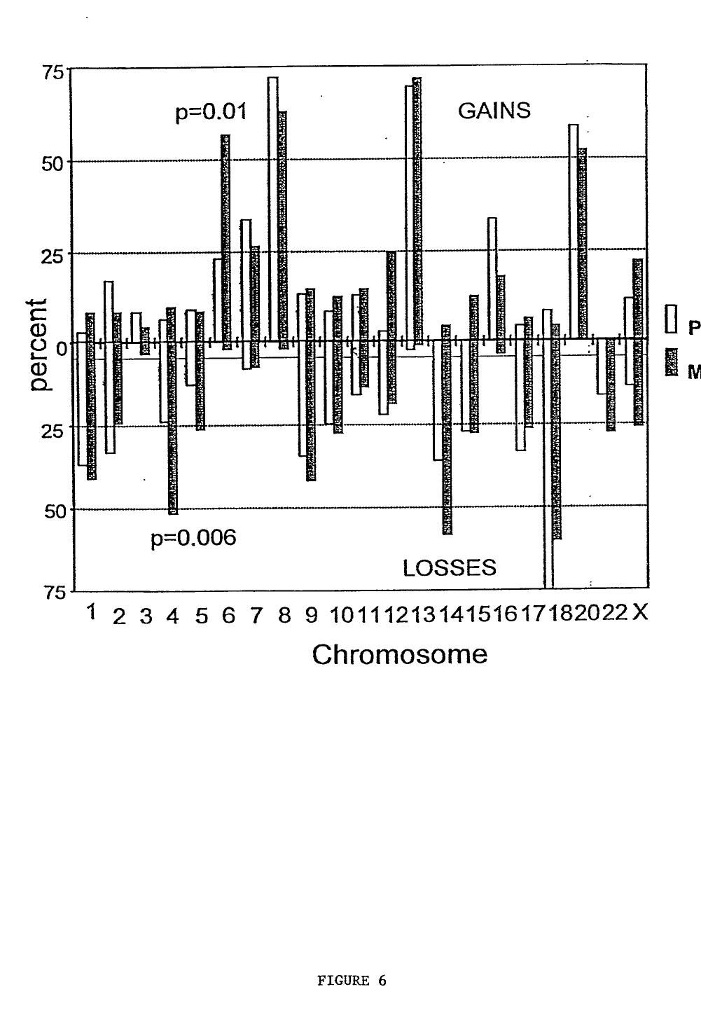

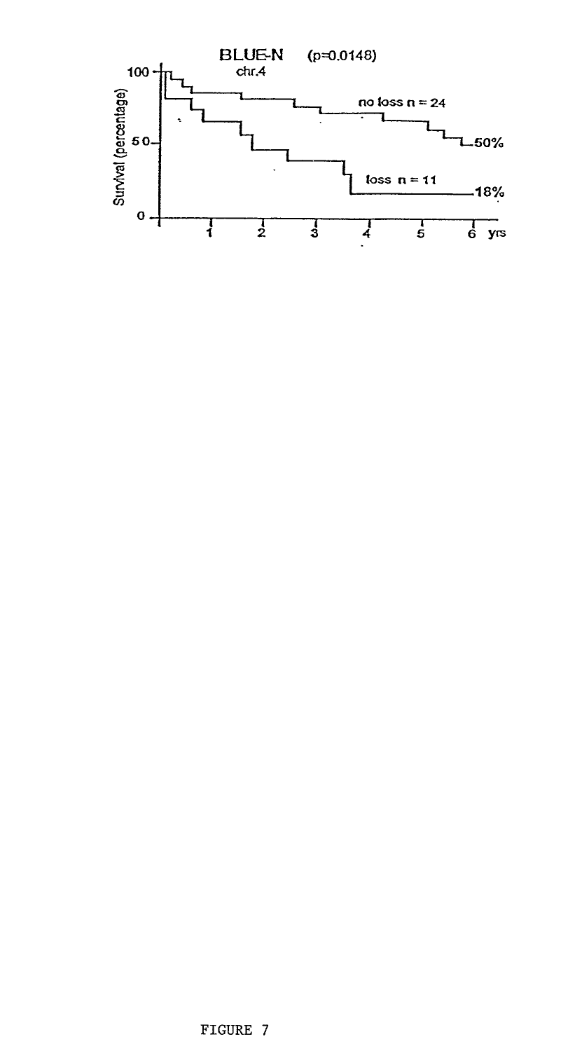

The symbols are as in FIG. 4. Percent indicate the average values of gains and losses from the multiple fingerprint bands for each chromosome (when appropriate, see FIG. 4) in the 12 primary (Dukes' D) and 25 metastatic tumors. The P values of the comparison between primary (P) and metastatic (M) tumors were calculated by the Fisher exact test. Only statistically significant values are shown (losses of chromosome 4 and gains of 6) with the exception of chromosome 12 gains (P=0.003). The P values considering only the 12 cases with primary and metastatic tumors from the same patients were P=0.024 for chromosome 4 losses, and P=0.057 and P=0.099 for chromosome 6 and 12 gains, respectively. Therefore, only the losses of chromosome 4 and gains of chromosome 6 are considered to be significantly increased in metastatic versus primary tumors.

FIG. 7. Prognostic value of AP-PCR fingerprinting for colon cancer.

The figure shows the survival curves of colorectal cancer patients according to the losses of a fingerprint band from chromosome 4. A panel of 35 colorectal carcinomas with follow-up information after surgical resections with curative intent (Arribas, R., Capella, G., Tortola, S., Masramon, L., Grizzle, W. E., Perucho, M. & Peinado, M. A., (1997) J. Clin. Oncol. 15, 3230-3240) were analyzed by AP-PCR DNA fingerprinting, and the differences in disease free survival time were compared relative to the alterations observed in the fingerprint bands. Kaplan-Meier disease free survival curves are plotted according to the allelic status of band N of the BLUE arbitrary primer fingerprints (Yasuda, J., Navarro, M., Malkhosyan, S., Velazquez, A., Seikya, T. & Perucho, M. (1996) Genomics 34, 1-7). Losses of this band are linked to increased risk, independently of Dukes stage (RR: 2.6, 95% CI: 1.0-6.4, p=0.0427).

### DETAILED DESCRIPTION OF THE INVENTION

In accordance with the present invention, methods are provided for determining the clinical outcome of a subject with a cancer using a Genomic Damage Fraction comprising,

a. determining the relative change in quantity of nucleic acids between cancerous cells and non-cancerous cells of said subject;

b. determining the Genomic Damage Fraction from the results of step (a);

c. determining the prognosis of said subject according to said subject's GDF, where a GDF greater than a predetermined GDF is indicative of a first clinical outcome (e.g., a poor prognosis), and a GDF lesser than a predetermined GDF is indicative of a second clinical outcome (e.g., a good prognosis).

For example, one aspect of this embodiment is where the relative change in quantity of nucleic acids is determined using AP-PCR DNA fingerprinting. Other aspects is of this embodiment is where the first clinical outcome is increased risk; where said second clinical outcome is decreased risk; where the relative change in nucleic acids is determined by the number of quantitative and/or qualitative changes in the DNA fingerprint bands present in the cancerous cells as compared with the normal cells; where the relative change in nucleic acids is determined by the number of quantitative changes in the DNA fingerprint bands; where the relative change in nucleic acids is determined by the number of qualitative changes in the DNA fingerprint bands; where the relative change in nucleic acids is determined by the number of quantitative and qualitative changes in the DNA fingerprint bands; where the relative change in nucleic acids is a gain or loss in quantity in nucleic acids; where the relative change in nucleic acids is a gain in quantity in nucleic acids; where the relative change in nucleic acids is a loss in quantity in nucleic acids; or where the subject with cancer has colorectal cancer.

Another embodiment of the present invention, is a method of determining the clinical outcome of a subject with a cancer comprising,

a. generating the AP-PCR DNA fingerprint of non-cancerous cells from said subject;

b. generating the AP-PCR DNA fingerprint of primary cancer cells from said subject;

c. generating the AP-PCR DNA fingerprint of metastatic cancer cells from said subject; and

d. identifying chromosomal regions from AP-PCR DNA fingerprint data of steps (a), (b) and (c) wherein the occurence of gains or losses of nucleic acids in certain chromosomal regions is prognostic of the clinical outcome for said subject.

For example, one aspect of this embodiment is where the gain or loss of nucleic acids is significantly different in metastatic cancer cells as compared to primary cancer cells; where the chromosomal region is determined by a band of chromosome 4 obtained using the BLUE primer (SEQ ID No: 1); or where the band is band N from the DNA fingerprint generated with the BLUE primer.

Still another embodiment of the present invention is a method of determining the clinical outcome of a subject with a cancer comprising,

a. generating the AP-PCR DNA fingerprint of non-cancerous cells from said subject;

b. generating the AP-PCR DNA fingerprint of primary cancer cells from said subject;

c. identifying chromosomal regions from AP-PCR DNA fingerprint data of steps (a) and (b), where gains or losses of nucleic acids occur; and

d. comparing said AP-PCR DNA fingerprints of chromosomes 1, 4, 6, 8, 9, and 13 from step a and step b wherein presence of gain or loss of nucleic acids in certain chromosomal regions is prognostic of the clinical outcome for said subject.

For example, one aspect of this embodiment is where the chromosomal region that is determined by band N of chromosome 4 from the Blue primer fingerprint is prognostic of the clinical outcome for said subject.

Yet another embodiment of the present invention is a method of predicting a clinical outcome of a subject with cancer using an amplotype from said subject comprising,

a. locating chromosomal regions that have gained and lost nucleic acids using AP-PCR DNA fingerprinting;

b. identifying said chromosomal regions that have lost nucleic acids; and

c. identifying said chromosomal regions that have gained nucleic acids;

wherein the combination of gains and losses according to chromosomal regions are prognostic of the clinical outcome for subject with cancer.

For example, one aspect of this embodiment is where the results of step (b) and step (c) are displayed where said gains and losses of nucleic acids are listed according to the chromosomal regions where they occur, wherein the combination of gains and losses according to chromosomal regions are prognostic of the clinical outcome for subject with cancer.

Definitions

As used herein, the term “Blue Primer” refers to the nucleic acid sequence, 5′ CCG AAT TCG CAA AGC TCT GA 3′ (SEQ ID NO: 1).

As used herein, the term “Genomic Damage Fraction”, or “GDF” refers to a measure of the change in quantity of nucleic acids between non-normal cells (e.g., cancerous cells) and normal cells in an individual. A predetermined GDF value is established by measuring the GDFs of a group of individuals with a cancer and correlating this information with actual clinical outcome for the individuals.

As used herein, the term “GDF_{G}” refers to a measure of the gain in quantity of nucleic acids between non-normal cells (e.g., cancerous cells) and normal cells in an individual.

As used herein, the term “GDF_{L}” refers to a measure of the loss in quantity of nucleic acids between non-normal cells (e.g., cancerous cells) and normal cells in an individual.

As used herein, the term “AP-PCR DNA fingerprinting” refers to a technique for the rapid generating of a set of discrete DNA amplification products characteristic of a genome as a fingerprint as described in U.S. Pat. No. 5,487,985 “McClelland '985”

As used herein, the term “amplotype” or “amplotyping” refers to the process of generating the AP-PCR DNA fingerprint of cancerous cells (e.g., primary cancer cells or metastatic cancer cells) and non-cancerous cells of a subject with cancer, and assembling the AP-PCR DNA fingerprint information according to gain and loss of nucleic acid material per chromosome where certain combinations of chromosomes gaining or losing nucleic acids is prognostic of the clinical outcome of the subject.

As used herein, the term “determining the clinical outcome of a subject with cancer” refers to whether the subject will have increased risk from the recurrence of the cancer, e.g., poor prognosis, such as, an increased rate of progress of a cancer in a subject, and/or the increased likelihood the cancer will become metastatic. Conversely, whether the subject will have decreased risk from recurrence of cancer, e.g., good prognosis, such as, a decreased rate of progress of a cancer in a subject, and/or a decreased likelihood the cancer will become metastatic.

Genomic instability characterizes the aneuploid cancer cell. Losses of genetic material are critical in cancer by exposing recessive mutations in tumor suppressor genes. Gains of genetic material may also lead to overexpression of genes contributing to tumor progression either in the presence or absence of mutation. However, the detection of moderate gains (such as tri-tetraploidy) has been a challenge in cancer research. Unbiased DNA fingerprinting by the Arbitrarily Primed PCR (AP-PCR) allows the detection moderate gains (in addition to losses) of DNA sequences of known chromosomal localization. Using AP-PCR DNA fingerprinting in this manner, a molecular karyotype of metastatic colon cancer is generated. This amplotype shows that sequences from several chromosomes undergo both losses (1, 4, 9, 14 and 18) and gains (6, 7, 12 and 20) in over half of the tumors. Moreover, gains of sequences from chromosomes 8 and 13 occurred in most tumors, indicating the existence in these chromosomes of positive regulators of cell growth or survival which are under strong positive selection during tumor progression. The over representation of these chromosomal regions is a critical step for metastatic colorectal cancer. Comparative amplotype analysis from primary and metastatic tumors shows the existence in chromosome 4 of gene(s) whose loss is specifically selected in cells that reach the metastatic stage.

Unbiased DNA Fingerprinting of Colorectal Cancer

AP-PCR DNA fingerprinting is applied to the analysis of chromosomal numerical changes in human colorectal cancer. Two arbitrary primers, MCG1 and BLUE, were selected based on their fingerprints quality (low background) and quantity (more than 25 bands). The chromosomal origin for most of the fingerprint bands was previously determined (Yasuda, J., Navarro, M., Malkhosyan, S., Velazquez, A., Seikya, T. & Perucho, M. (1996) Genomics 34, 1-7). Each autosome was represented by at least one fingerprint band, except chromosomes 18, 19 and 21. Therefore, estimation of band losses and gains of the fingerprints generated by these two primers allowed to establish a molecular karyotype of colorectal cancer. This molecular karyotype is called an “amplotype”, to distinguish it from the conventional “allelotype”, whereby only LOH, and by inference allelic losses, can be determined. The metastatic tumors are analyzed to determine the amplotype with the presumed highest number of chromosomal changes.

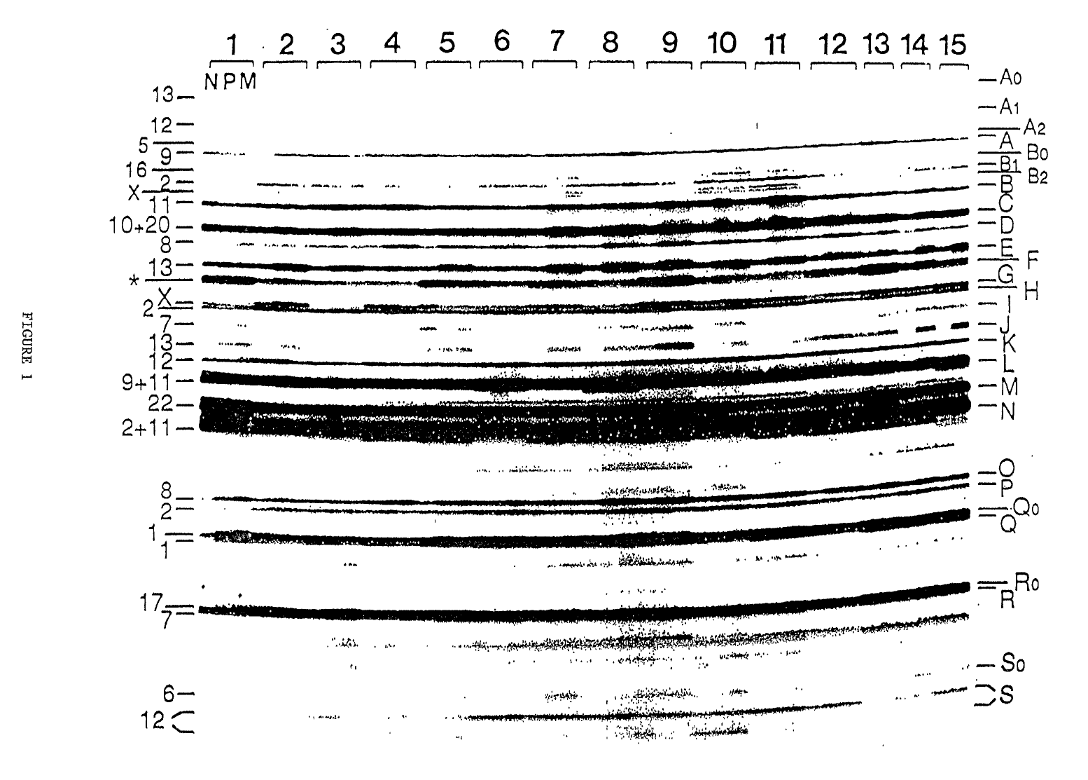

FIG. 1 shows the AP-PCR fingerprints generated by the arbitrary primer MCG1. Differences in band intensity are frequent in the DNA fingerprints from normal versus tumor tissues. Some of these differences are due to variation in the overall levels of amplification between DNAs (compare the backgrounds of cases 10, 12 and 14) and are not considered significant (for instance the increased intensity of band A in the metastasis of case 14). The intensity changes of other bands are on the other hand reproducible (see Examples for the criteria followed for scoring gains and losses). Because of the complexity of the figure, we describe only some of the representative bands: D and O, derived from chromosome 8, and E and J, derived from chromosome 13. These bands showed consistent increases in tumor tissue DNAs, in contrast with other bands exhibiting sporadic intensity changes, such as the increased intensity of band A in cases 2 and 7 in both primary (P) and metastatic (M) tumors, and case 8 (only M). Examples of increases in bands E and J: cases 1 (only band J, in both P and M); 2, 7 and 8 (both bands E and J, in both P and M); 3 (only band E, in both P and M). Examples of increases in bands D and O: cases 4, 5, and 12 (both bands D and O, in both P and M); 8, 9, 10 and 11 (band O, in both P and M; band D, in P); and cases 13 and 14 (both bands D and O).

Application of the Genomic Damage Fraction

The “Genomic Damage Fraction”, or “GDF” is a measure of the change in quantity of nucleic acids between non-normal cells (e.g., cancerous cells) and normal cells in an individual. Typically, the GDF is used to compare normal cells with tumor cells, and also, normal cells with primary cancer cells and metastatic cancer cells. For example, the GDF can be derived using AP-PCR DNA fingerprinting techniques. The AP-PCR technique is used to generate DNA fingerprints of normal and non-normal cells. The DNA fingerprints are presented in the form of bands of nucleic acid materials representative of certain chromosomal regions for the subject as resolved by electrophoresis gel, e.g., as depicted in FIG. 1. The DNA fingerprint of normal cells is by definition the baseline standard. Generally, the DNA fingerprint of the cancerous cells will be different from that of non-cancerous cells, where the cancerous cells will have additional and/or stronger bands (i.e., gain of nucleic acid material) as compared with the non-cancerous cells, or missing and/or weaker bands (i.e., loss of nucleic acid material) as compared with the non-cancerous cells. The total number of deviations, i.e., number of occurrences of additional and/or stronger bands and of missing and/or weaker bands, is divided by the total number of bands present in the non-cancerous cells resulting in the GDF.

A measure of the gain in nucleic acids in the non-normal cells can be generated from the number of bands corresponding to gained nucleic acid is divided by the total number of bands present in the non-cancerous cells resulting in the GDF_{G}.

A measure of the loss in nucleic acids in the non-normal cells can be generated from the number of bands corresponding to lost nucleic acid is divided by the total number of bands present in the non-cancerous cells resulting in the GDF_{L}.

The intensity of the bands vary with the density of nucleic acids aggregating at a given position on an electrophoresis gel. A band with stronger intensity in the fingerprint from tumor tissue DNA indicates the existence of more DNA fragments in the fingerprint gel and by extrapolation, of more DNA molecules in the donor tumor tissue cells relative to the normal tissue cells, for example, by gains of nucleic acids in the region of the tumor cell genomes corresponding to the sequences of the particular fingerprint band. Conversely, a fainter band in the tumor tissue fingerprint represents the loss of the corresponding genomic sequences. A complete loss of a particular sequence, for example, a homozygous deletion, is reflected by the absence of the band. While a partial loss of the sequence, for example, a heterozygous deletion—loss of one of the two alleles of a particular sequence, is reflected by a fainter band. Similarly, a gain of a few copies of a particular allelic sequence will be reflected in a band of moderately stronger intensity in the tumor fingerprint, for example, double or triple intensity. A gain of many copies of a particular sequence will be reflected by a more drastic increase in intensity. When this occurs with a genomic sequence that generates a very faint band in the normal tissue fingerprint, the amplification of the sequence in the tumor cell genome may appear as a new band in the tumor fingerprint. Both stronger and weaker fingerprint bands represent therefore relative quantitative changes in nucleic acids content between tumor and normal tissues.

Qualitative changes in APPCR fingerprints are on the other hand changes that are due to structural alterations in the genome of the tumor cell. Thus, a chromosomal rearrangement such as a translocation, or a deletion or a insertion of a particular segment of the genome may result in a new band in the fingerprint. Examples of such qualitative alterations are the deletion mutations that are very common in tumors of the microsatellite mutator phenotype (Ionov, Y., Peinado, M. A., Malkhosyan, S., Shibata, D. & Perucho, M. (1993) Nature (London) 363, 558-561). These ubiquitous deletion mutations in simple repetitive sequences or microsatellites are reflected by a change in the mobility of some fingerprint bands (i.e., a new band appears in the tumor fingerprint). The GDF may or may not incorporate these qualitative changes.

Not all bands are of the same intensity in each fingerprint, some bands are more intense than others. Therefore, the actual number of bands discernable and hence recorded will vary according to the degree of sensitivity for detection. Although this is potentially a source of variability in the measure from practitioner to practitioner, as long as the degree of detection is applied consistently by each practitioner to designate whether a band is present or absent, the ultimate value of GDF will be consistent, because any differences between practitioners is negated by virtue of the GDF being a ratio of the change in number bands and total number of bands.

It should also be noted that the GDF is normalized for differences within a given individual and therefore can be used as a measure between different individuals, or populations of individuals. As such the GDF can be used as a quantitative measure of the change in quantity of nucleic acids between normal and non-normal cells in an individual. GDF may also be used as a qualitative measure between a first individual and a second individual, or first individual and a population of individuals. For this reason, GDF is a useful tool for epidemiological studies of diseases that are associated with changes in the quantity of nucleic acids in individuals.

GDF provides a quantitative measure of the genomic damage evidenced in cancer cells against non-cancer cells in a subject. The GDF can also provide a qualitative measure of a subject's survivability when the subject's GDF is compared with an established GDF value, e.g., where a subject with a GDF higher than an established value is indicative of greater risk for the recurrence of the disease, and a GDF lower than the established value is indicative of lesser risk for the recurrence of the disease.

Allelic Losses and Gains in Colorectal Cancer

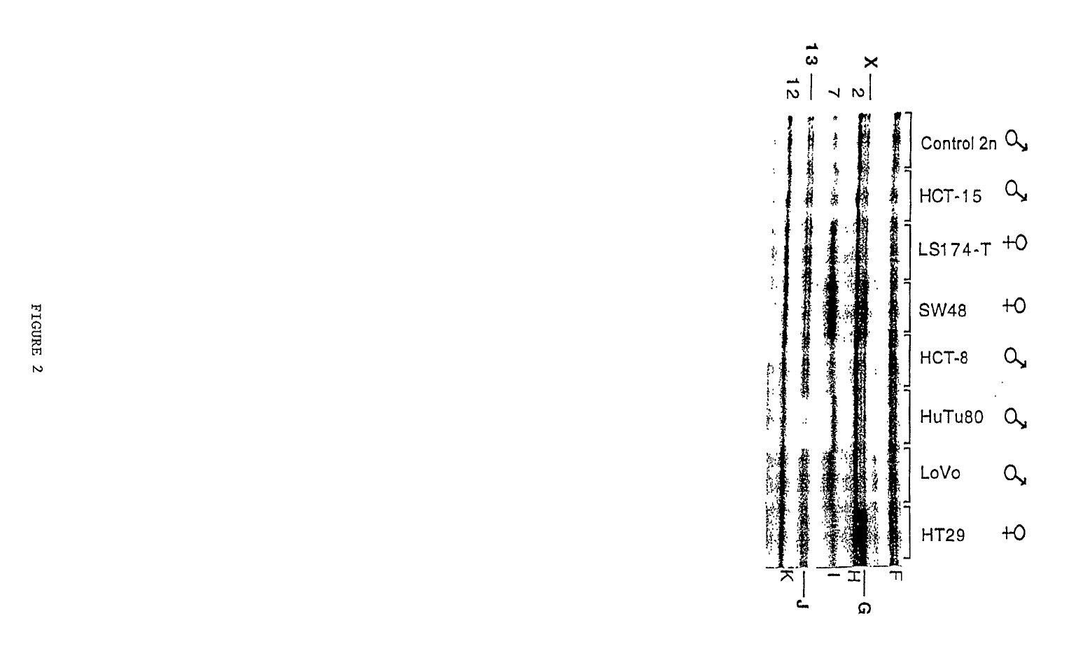

Southern blot hybridization experiments with cloned bands E, J and D showed that tumor-specific increased intensity of these fingerprint bands was due to the higher copy number of target sequences, and not an artifact of the in vitro amplification (Peinado, M. A., Malkhosyan, S., Velazquez, A & Perucho, M. (1992) Proc. Natl. Acad. Sci. USA 89, 10065-10069 data not shown). The quantitative nature of the amplification levels is also illustrated by the concordance of the relative intensities of band G from chromosome X with the gender of the cancer patients (FIG. 1). The ability of AP-PCR DNA fingerprinting to detect moderate changes of chromosome copy number is also shown in the fingerprints of tumor cell lines of known karyotype (FIG. 2). In addition to the correspondence of chromosome X band intensity with the gender of the donors, loss of an autosome is reflected by the decreased intensity of band J from chromosome 13 in the HuTu80 cell line, monosomic for this chromosome. The intensity variation of band gains (see Examples) ranged from 2.276+/−1.27 in tumor DNA in the case with higher level of gains (chromosome 13 band E), which we estimate represents 3 to 7 copies.

Ambiguity of Microallelotyping for Determining the Nature of Allelic Imbalances

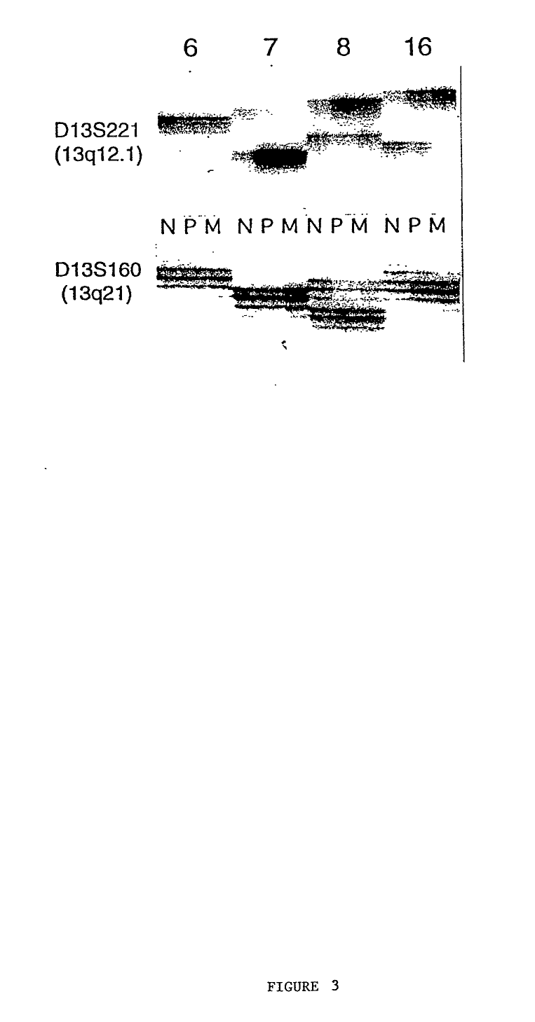

We carried out PCR amplification of two chromosome 13 microsatellite loci (FIG. 3) to determine the relationship between the chromosomal imbalances detected by fingerprinting and by microsatellite analysis, commonly used for the estimation of LOH in tumors (microallelotyping). The results show that microallelotyping may be misleading to interpret the allelic composition of the loci analyzed. Thus, in tumors 7, 8 and 16, the results can be interpreted as indicative of LOH at the D13S221 locus and in tumors 8 and 16 of the D13S160 locus. However, as shown by the fingerprints (FIG. 1), in concert with Southern blot hybridization (data not shown), the consistent change in these tumors was the gain but not the loss of chromosome 13 sequences. These results show that microallelotyping only detects allelic imbalances because an apparent loss of one allele in tumor compared to normal tissue can be due to the gains of the other allele.

While the use of microalleltyping alone is insufficient to determine gains, in combination with amplotyping provides additional information on the chromosomal alterations undergone in the aneuploid cancer cell. For instance, while in the telomeric chromosome 13 region both alleles appear to be gained, in the more centromeric 13q14-q21 region, gains of one allele are accompanied by the losses of the other (FIG. 4).

Amplotype of Colorectal Cancer

The global results of these analyses are represented in FIG. 5. The frequency of losses and gains was similar. An average of near 25% of all chromosomal regions analyzed exhibited losses or gains per tumor and their combined values reached near 50%. The average frequency of chromosomal losses is slightly higher than the fractional allelic loss (FAL) determined by allelotyping (Vogelstein, B., Fearson, E. R., Kern, S. E., Hamilton, S. R., Preisinger, A. C., Kakamura, Y. & White, R. (1989) Science 244, 217-221). This may be explained because of the more advanced stage of progression of our tumors. On the other hand, the amplotype approach is not as sensitive to detect allelic losses as the allelotype procedure because loss of one allele and reduplication of the other would score positive by allelotyping but negative by amplotyping. This situation is revealed by the combination of fingerprinting, Southern blot and microallelotyping. For instance, case 7, shows that the intensity of the D band in the fingerprint is not decreased (FIG. 1). However, allelotyping of these bands showed the loss of one allele of the chromosome 8 D band in both primary and metastatic tumor (data not shown), indicating that the loss of one allele was accompanied by the reduplication of the other. Similar conclusion can be reached for the length polymorphic band S from chromosome 12 in both tumor tissues of case 9 (FIG. 1). The gain of the long allele compensates the loss of the short.

The amplotype of metastatic colon cancer (FIG. 5) shows that losses of sequences from chromosomes 1, 4, 9, 14 and 18 occurred in about 50% of the tumors. Over 50% of tumors also exhibited gains of bands from chromosomes 6 and 20 and over 75% of tumors exhibited gains in multiple bands from chromosomes 8 and 13.

Chromosomal Imbalances in Primary and Metastatic Colon Cancer

The availability of 12 cases with both primary and metastatic tumors allowed to investigate whether any of the chromosomal changes were metastasis-specific. Most consistent gains or losses were not significantly associated with metastatic cancer. For instance, neither the gains of chromosome 8 or 13 were events specific for the metastatic process, but to precede it. However, chromosome 4 losses and chromosome 6 gains were significantly associated to the metastatic stage (FIG. 6).

AP-PCR DNA fingerprinting can be used to detect genetic alterations during tumorigenesis. This approach presents several advantages compared with other techniques. One PCR reaction allows to quantitatively compare normal and tumor tissues at multiple sites of the genome and also permits the single-step cloning of DNA fragments representing altered genomic sites (Peinado, M. A., Malkhosyan, S., Velazquez, A & Perucho, M. (1992) Proc. Natl. Acad. Sci. USA 89, 10065-10069). It also gives an overall picture of the extent of genetic damage in tumor cells which may have prognostic value for cancer (Arribas, R., Capella, G., Tortola, S., Masramon, L., Grizzle, W. E., Perucho, M. & Peinado, M. A. (1997) J. Clin. Oncol. 15, 3230-3240). The technique also permits the simultaneous identification in many tumor samples of moderate allelic losses and gains. The genomic localization of these loci can be previously determined at the chromosomal or subchromosomal levels by the use of somatic human-rodent monochromosome or radiation hybrids (Peinado, M. A., Malkhosyan, S., Velazquez, A & Perucho, M. (1992) Proc. Natl. Acad. Sci. USA 89, 10065-10069; and Yasuda, J., Navarro, M., Malkhosyan, S., Velazquez, A., Seikya, T. & Perucho, M. (1996) Genomics 334, 1-7 and work in progress). Consequently, this approach represents a molecular tool of high resolution for cancer cytogenetics.

Among the novel findings in this study is the existence of potential new tumor suppressor genes for colorectal cancer at chromosomes 1 and 9 and for the metastatic stage at chromosome 4. There are no reports of recurrent chromosome 4 losses nor of metastasis-specific cancer genes in colorectal cancer. This underscores the power of unbiased DNA fingerprinting to identify new genomic regions containing potential tumor suppressor genes. In addition, losses of one of the bands from chromosome 4 have value as prognostic indicator for increased risk to colorectal cancer. Independent AP-PCR analysis of a collection of colorectal carcinomas for which follow-up information was available (Arribas, R., Capella, G., Tortola, S., Masramon, L., Grizzle, W. E., Perucho, M. & Peinado, M. A. (1997) J. Clin. Oncol. 15, 3230-3240) revealed that among the fingerprint band displaying recurrent alterations, somatic loss of the Blue primer band N from chromosome 4 (Yasuda, J., Navarro, M., Malkhosyan, S., Velazquez, A., Seikya, T. & Perucho, M. (1996) Genomics 34, 1-7) was a prognostic indicator of poor survival (FIG. 7). Therefore, the association of the losses of these sequences with the metastatic stage may be explained by assuming that the tumors that undergo the losses of these sequences have an enhanced ability to metastasize.

Thus, another embodiment of the present invention is a method of identifying genomic regions relevant for cancer. The method is exemplified by the experiment described in FIG. 1. It is performed in a single denaturing gel electrophoresis by PCR amplification with a single arbitrary primer of the DNA from normal, primary and metastatic tissues of a panel of cancer patients, yielding relevant information for cancer diagnosis and prognosis, for example as exemplified in the amplotype of FIG. 5. In combination with follow-up information of the corresponding cancer patients, (e.g., continued monitoring of the physical progression or regression of the tumor, the recurrence of the tumor after either surgical intervention or radiation therapy, development of other related or unrelated tumors in the patient, and the like) the task of finding prognostic markers for cancer is facilitated by focusing on only the minority of sequences that are likely to be useful prognostic markers (i.e., those fingerprint bands tightly linked to metastatic-specific cancer genes). While the procedure does not identify the actual responsible cancer gene (i.e., the metastatic gene), it identifies a genomic region that is closely linked to the cancer gene, thus facilitating the subsequent task of gene hunting.

While the specific example of the invention is a band undergoing frequent losses in metastatic but not primary cancers, other situations can be envisioned where the prognostic marker will be the gain of a specific sequence more frequently in metastatic than in primary cancers.

In addition, once identified the relevant prognostic marker, and its rapid isolation from the fingerprint gels provides a further simplification of the experimental approach of generating useful cancer prognostic markers. For example, mapping of the band to the chromosomal region at 4pl6 allows one of ordinary skill in the art to immediately identify other close polymorphic markers in the same chromosomal region, such as, D4S339 and D4S524 (The Genome Database (http://gdbwww.gdb.org/). This then facilitates the task of the screening of tumors for losses of this chromosomal region by the standard microallelotyping approach. Detection of losses of heterozygosity (LOH) in any of these adjacent dinucleotide microsatellite markers may be also useful for cancer prognosis, and more amenable to routine testing in clinical settings.

The other novel finding of our study is that moderate gains of chromosome sequences are equally prevalent as the losses. While determination of losses is “digital” (yes or no), the extent of chromosomal sequence gains represented by the fingerprint bands is “analogical”, since in principle there is no upper limit. However, we estimate that these gains represented no more than 5-7 copies in any of the cases studied with the arbitrary primers used in this work. Detection of typical amplicons (undergoing more than 10 fold amplification) by AP-PCR DNA fingerprinting can be achieved by using more primers (Okazaki, T., Takita J., Kohno, T., Handa, H. & Yokota, J. (1996) Hum. Genet. 98, 253-258). The lower limit of detection of chromosomal gains appears to be 3, but due to sensitivity limitations of the method, triploidy cannot formally be distinguish from tetraploidy. However, in contrast with the “digital” losses, the functional difference of an “analogical” gain of three versus four copies seems not so critical.

We also found that gains of some chromosomes occur with a frequency significantly higher than previously reported by cytogenetic and molecular cytogenetic (CGH) approaches. This can be explained because our approach to detect gains is independent of the location of the gained sequences either in a chromosome recognizable cytogenetically, or within a chromosomal region of a minimum size to be detectable by CGH. The high frequency of moderate gains of chromosomes 6, 8 and 13, illustrates the importance for tumor progression of chromosomal imbalances leading to moderate over representation of gene products.

It has been shown that DNA fingerprinting by AP-PCR fulfills the requirements for a technique, that because of its simplicity and sensitivity, it facilitates the estimation of the prevalence of moderate gains of genetic material in tumors. These gains imply the existence of a gene or a set of genes in the corresponding chromosomes whose moderate gains are selected during tumor progression, probably because their products confer a selective advantage for growth or survival to the tumor cells. This hypothesis is based on the principle that if the same somatic mutation occurs independently and in a consistent manner, it is probably relevant for tumor development (Baker, S. J., Fearon, E. R., Nigro, J. M., Hamilton, S. R., Preisinger, A. C., Jessup, J. M., van Tuinen, P., Ledbetter, D. H., Barker, D. F., Nakamura, Y., et al. (1989) Science 244, 217-221; and Perucho, M., Goldfarb, M., Shimizu, K., Lama, C., Fogh, J. & Wigler, M. (1981) Cell 27 467-476).

Gains of chromosome 8q have been described in colorectal cancer by cytogenetics and molecular cytogenetics. The frequency of such gains, often due to trisomy, range from 20-50% by cytogenetics: 9 of 18 (Muleris, M., Salmon, R. J. & Dutrillaux, B. (1990) Cancer Genet. Cytogenet. 46 143-156); 36 of 116 (Bardi, G., Sukhikh, T., Pandis, N., Fenger, C., Kronborg, O. & Heim, S. (1995) Genes Chromosomes Cancer 12, 97-109); or by CGH: 8 of 16 (Ried, T., Knutzen, R., Steinbeck, R., Blegen, H., Schrock, E., Heselmeyer, K., du Manoir, S. & Auer, G., (1996) Genes Chromosomes Cancer 15, 234-245). Using AP-PCR fingerprinting there is a frequency of gains of 76% by scoring individual fingerprint bands (FIG. 5). These three fingerprint bands have been localized to 8 q subchromosomal regions (at a resolution of a few megabases) by the use of radiation hybrid panels (manuscript in preparation). The data does not excludes c-Myc as the most obvious candidate for the 8q gene selected for during tumor progression. There are numerous reports on c-Myc amplification in colon cancer (Marcu, K. B., Bossone, S. A. & Patel, A. J. (1992) Annu. Rev. Biochem. 61, 809-860 and references wherein). However, none of these reports reached a frequency as high as that obtained by amplotyping, probably due to the difficulties in determining moderate levels (equivalent to trisomy/tetrasomy) of gains by molecular hybridization approaches. Therefore, the role of c-Myc activation in colorectal cancer might be underestimated. Nevertheless, c-Myc may not be the only functional locus in the 8q gains, and the chromosomal region undergoing over representation may contain additional relevant genes for tumor progression (Okazaki, T., Takita, J., Kohno, T., Handa, H. & Yokota, J. (1996) Hum. Genet. 98, 253-258).

Similar analysis of the chromosome 13 gains yields a more complex pattern, because the three fingerprint bands A₀, E and J are concomitantly over represented in many cases (FIG. 4). Work in progress is aimed to localize more precisely the common region(s) of gain of this chromosome, which have been also previously implicated in colon cancer by cytogenetic: 10 of 18 (Muleris, M., Salmon, R. J. & Dutrillaux, B. (1990) Cancer Genet. Cytogenet. 46 143-156); 37 of 116 (Bardi, G., Sukhikh, T., Pandis, N., Fenger, C., Kronborg, O. & Heim, S. (1995) Genes Chromosomes Cancer 12, 97-109); RFLP/LOH: 10 of 31 (Lothe, R. A., Fossli, T., Danielsen, H. E., Stenwig, A. E., Nesland, J. M., Gallie, B. & Borresen, A. L., (1992) J. Natl. Cancer Insti. 84, 1100-1108); and CGH: 8 of 16 (Ried, T., Knutzen, R., Steinbeck, R., Blegen, H., Schrock, E., Heselmeyer, K., du Manoir, S. & Auer, G., (1996) Genes Chromosomes Cancer 15, 234-245) and 5 of 12 (Schlegel, J., Stumm, G., Scherthan, H., Bocker, T., Zirngibl, H., Ruschoff, J. & HOfstadter, F. (1995) Cancer Res 55, 6002-6005) analyses, but with lower frequency. Because of this high incidence, the over representation of 13q loci is a nearly obligatory step for the late stages of colorectal cancer.

It is also shown that microallelotyping by PCR amplification of dinucleotide repeats exhibits an intrinsic ambiguity on the determination of losses or gains of the polymorphic alleles, which may be misleading to identify the relevant alteration (FIG. 3). Thus, microallelotyping may be only informative to determine allelic imbalances (Ah-See, K. W., Cooke, T. G., Pickford, I. R., Soutar, D. & Balmain, A. (1994) Cancer Res 54, 1617-1621) but not LOH (Nawroz, H., van der Riet, P., Hruban, R. H., Koch, W., Ruppert, J. M. & Sidransky, D., (1994) Cancer Res. 54 1152-1155) which are not equivalent. However, in concert with DNA fingerprinting, microallelotyping may add critical information on the mechanisms underlying these chromosomal alterations. Our results indicate that at least in some cases, the relevant alteration is the quantitative increase in genetic material, because there appear to be a selection for gains of either of the two alleles. For instance, in some cases, one of the chromosome 13q alleles is over represented in the primary tumor, but the other in the metastasis (FIG. 4). In these situations, the gained chromosome probably harbors wild type allele(s). On the other hand, gains of some sequences may be also accompanied by LOH (for instance, see FIG. 1 case 9, band S; FIG. 4 and (Achille, A., Biasi, M. O., Zamboni, G., Bogina, G., Magalini, A. R., Pederzoli, P., Perucho, M. & Scarpa, A., (1996) Cancer Res. 56, 3808-3813), suggesting the co-existence of positive and negative regulators of cell growth or survival closely located in the same chromosomal region. These findings may be disclosing a protective mechanism for cancer development, because it would hinder the mutational unmasking of the oncogenic potential latent in these chromosomal regions.

The following examples are given to enable those of ordinary skill in the art to more clearly understand and to practice the present invention. The examples should not be considered as limiting the scope of the invention, but merely as be illustrative and representative thereof.

### EXAMPLES

### Example 1

### Method of Identifying Genes Associated with Colorectal Cancer

Tumor Samples

Primary colorectal tumors their normal tissue counterparts included 12 primary Dukes' D colon carcinomas and 25 colon cancer liver metastases (12 of these samples were derived from the same 12 primary carcinoma patients). Most of these tumors were obtained from the National Cancer Center at Tokyo. In agreement with the low incidence of metastasis in colon cancer of the microsatellite mutator phenotype (Ionov, Y., Peinado, M. a., Malkhosyan, S., Shibata, D. & Perucho, M. (1993) Nature (London) 363, 558-561), none of the tumors studied exhibited enhanced microsatellite instability. Some metastatic colorectal carcinomas and a panel of 80 tumors at earlier stages of tumor progression with follow-up data (Arribas, R., Capella, G., Tortola, S., Masramon, L., Grizzle, W. E., Perucho, M. & Peinado, M. A. (1997) J. Clin. Oncol. 15 3230-3240) were obtained from the Human Tissue Cooperative Network (University of Alabama, Birmingham).

AP-PCR DNA Fingerprinting

Genomic DNA was prepared from tumor and normal tissues as described (Peinado, M. A. Malkhosyan, S., Velazquez, A., & Perucho, M., (1992) Proc. Natl. Acad. Sci. USA 89, 10065-10069; and Arribas, R., Capella, G., Tortola, S., Masramon, L., Grizzle, W. E., Perucho, M. & Peinado, M. A. (1997) J. Clin. Oncol. 15, 3230-3240. DNA (50-100 ng) was subjected to AP-PCR amplification in 25 ml of reaction mix: 1 unit of Taq DNA polymerase (Perkin-Elmer-Cetus), 10 mM of Tris-HCl (pH 8.3), 50 mM of KCl, 4.5 mM of Mg Cl2, 0.1% gelatin, and 1 mM of primer. The AP-PCR conditions were as previously described in (Peinado, M. A. Malkhosyan, S., Velazquez, A., & Perucho, M., (1992) Proc. Natl. Acad. Sci. USA 89, 10065-10069) with 25 high stringency cycles. The number of cycles is an important parameter to maintain linearity of amplification and is determined empirically for each primer. The PCR products were electrophoresed in a 5.5% polyacrylamide gel (Peinado, M. A. Malkhosyan, S., Velazquez, A., & Perucho, M., (1992) Proc. Natl. Acad. Sci. USA 89, 10065-10069) at 55 W for 5 to 6 hours. The MCG1 and BLUE arbitrary primer's sequences have been described in (Yasuda, J., Navarro, M., Malkhosyan, S., Velazquez, A., Seikya, T. & Perucho, M. (1996) Genomics 34, 1-7). The sequence of the F primer is 5′ ATT CAA GAC TGC CTT TCC TA 3′.

Chromosomal Assignment of AP-PCR Fingerprint Bands

Chromosome assignment was determined by PCR of monochromosome human- rodent cell hybrids NIGMS panels 1 and 2 (Coriell Cell Research) using specific primer sets previously designed based on the sequence of the cloned fragments. Bands were extracted with 100 ml of distilled water and reamplified with the same arbitrary primer and cloned using the PCR script system (Stratagene) and the TA cloning system (Invitrogen) following the manufacturers' instructions. To confirm the authenticity of the cloned bands, they were used as probes in Southern blots of AP-PCR gels (Perucho, M., Welsh, J., Peinado, M. A., Ionov, Y. & McClelland, M. (1995) Methods Enzymol. 254, 275-290). Correct clones were selected by comparison of the fingerprint and blotting patterns. The chromosomal origin of other fingerprint bands was determined by the SHARP method (Yasuda, J., Navarro, M., Malkhosyan, S., Velazquez, A., Seikya, T. & Perucho, M. (1996) Genomics 34, 1-7). Genomic Southern blot analysis of cloned AP-PCR bands was done as described (Peinado, M. A. Malkhosyan, S., Velazquez, A., & Perucho, M., (1992) Proc. Natl. Acad. Sci. USA 89, 10065-10069).

Densitometrical Analysis

The dried gels were exposed to X-ray film at room temperature without image intensifier or at −70° C. with image intensifier at different time exposures. Autoradiograms were scanned with ImageMaster Desk Top Scanner (Pharmacia LKB). The scanned image was analyzed with ImageMaster software (Pharmacia) using an IBM-PC PS/V personal computer. The backgrounds of the X-ray film and of the individual lanes were subtracted using the Rolling-Disk background subtraction method following the instructions by the manufacturer. Film calibration was made by serial dilution of ³²P-labeled oligonucleotides, and the radioactivity was standardized by Cerenkov counting using LS3801 liquid scintillation system (Beckman), with the same exposure conditions than the AP-PCR fingerprinting gels.

Determination of the Status of Gains and Losses

Scoring quantitative changes between normal and tumor tissue fingerprint bands was made by densitometrical analysis and by visual inspection. To establish the criteria for gain and loss in the densitometrical analysis, the data of the fingerprints of normal samples were calibrated. The mean standard deviations of non-polymorphic bands were estimated to be around 10%. In other words, the range of fluctuations in band intensity due to experimental variation usually was between 0.9 to 1.1. Considering the contamination of tumor tissue by normal cells, a normal range was established of apparent allelic variation from 0.75 to 1.25 of the tumor/normal ratio. Therefore, only fluctuations in band intensity superior to this range were considered diagnostic of chromosomal imbalances. Even with 50% of normal tissue DNA contamination in tumor DNA, the ratio of trisomy (in a hypothetical cell retaining diploidy in the rest of the chromosomes) would appear as 1.25 and the ratio of the loss of one allele would appear as 0.75. Our tumors did not have more than a 20-30% contamination of normal tissue, as analyzed by histological examination (data not shown). Therefore, the approach used in the scoring of gains and losses has been conservative.

We claim:

1. A method of determining the clinical outcome of a subject with a cancer using a Genomic Damage Fraction comprising,
a. determining the relative change in quantity of nucleic acids between cancerous cells and non-cancerous cells of said subject;
b. determining the Genomic Damage Fraction from the results of step (a)
c. determining the prognosis of said subject according to said subject's GDF, where a GDF greater than a predetermined GDF is indicative of a first clinical outcome, and a GDF lesser than a predetermined GDF is indicative of a second clinical outcome.

2. The method of claim 1, wherein the relative change in quantity of nucleic acids is determined using AP-PCR DNA fingerprinting.

3. The method of claim 1, wherein said first clinical outcome is increased risk.

4. The method of claim 1, wherein said second clinical outcome is decreased risk.

5. The method of claim 1, wherein the relative change in nucleic acids is determined by the number of qualitative and/or quantitative changes in the DNA fingerprint bands present in the cancerous cells as compared with the normal cells.

6. The method of claim 5, wherein the relative change in nucleic acids is determined by the number of quantitative changes in the DNA fingerprint bands.

7. The method of claim 5, wherein the relative change in nucleic acids is determined by the number of qualitative changes in the DNA fingerprint bands.

8. The method of claim 5, wherein the relative change in nucleic acids is determined by the number of quantitative and qualitative changes in the DNA fingerprint bands.

9. The method of claim 1, wherein the relative change in nucleic acids is a gain in quantity in nucleic acids.

10. The method of claim 1, wherein the relative change in nucleic acids is the combination of gain and loss in quantity in nucleic acids.

11. The method of claim 1, wherein the relative change in nucleic acids is a gain in quantity in nucleic acids.

12. The method of claim 1, wherein the subject with cancer has colorectal cancer.

13. A method of determining the clinical outcome of a subject with a cancer comprising,
a. generating the AP-PCR DNA fingerprint of non-cancerous cells from said subject;
b. generating the AP-PCR DNA fingerprint of primary cancer cells from said subject;
c. generating the AP-PCR DNA fingerprint of metastatic cancer cells from said subject; and
d. identifying chromosomal regions from AP-PCR DNA fingerprint data of steps (a), (b) and (c) wherein the occurrence of gains or losses of nucleic acids in certain chromosomal regions is prognostic of the clinical outcome for said subject.

14. The method of claim 13, wherein the gain and loss of nucleic acids is significantly different in metastatic cancer cells as compared to primary cancer cells.

15. The method of claim 13, wherein said chromosomal region is determined by a band of chromosome 4 obtained using the Blue primer (SEQ ID No: 1).

16. The method of claim 15, wherein said band is band N from the DNA fingerprint generated with the Blue primer (SEQ ID. NO:1).

17. A method of determining the clinical outcome of a subject with a cancer comprising,
a. generating the AP-PCR DNA fingerprint of non-cancerous cells from said subject;
b. generating the AP-PCR DNA fingerprint of primary cancer cells from said subject;
c. identifying chromosomal regions from AP-PCR DNA fingerprint data of steps (a) and (b), where gains or losses of nucleic acids occur; and
d. comparing said AP-PCR DNA fingerprints of chromosomes 1, 4, 6, 8, 9, and 13 from step a and step b wherein presence of gain or loss of nucleic acids in certain chromosomal regions is prognostic of the clinical outcome for said subject.

18. The method of claim 17 wherein said chromosomal region that is determined by band N of chromosome 4 from the BLUE primer (SEQ ID NO: 1) fingerprint is prognostic of the clinical outcome for said subject.

19. A method of predicting a clinical outcome of a subject with cancer using an amplotype from said subject comprising,
a. locating chromosomal regions that have gained and lost nucleic acids using AP-PCR DNA fingerprinting;
b. identifying said chromosomal regions that have lost nucleic acids; and
c. identifying said chromosomal regions that have gained nucleic acids;
wherein the combination of gains and losses according to chromosomal regions are prognostic of the clinical outcome for subject with cancer.

20. The method of claim 19, wherein the results of step (b) and step (c) are displayed where said gains and losses of nucleic acids are listed according to the chromosomal regions where they occur, wherein the combination of gains and losses according to chromosomal regions are prognostic of the clinical outcome for subject with cancer.

21. A method of identifying a genomic region relevant for a cancer in a subject having said cancer comprising,
(a) generating the AP-PCR DNA fingerprint of non-cancerous cells, primary cancer, and metastatic tumor cells from said subject; and
(b) identifying said genomic regions from AP-PCR DNA fingerprint data of step (a), showing gains and losses of nucleic acids is certain genomic regions thereby identifying a genomic region linked to a cancer gene.

22. The method of claim 21, wherein said cancer is colorectal cancer.

23. The method of claim 21, wherein the said AP-PCR DNA fingerprint is generated with the Blue primer (SEQ ID NO: 1).
